(19) United States
(12) Patent Application Publication
(10) Pub. No.: US 2012/0022094 A1
(43) Pub. Date: Jan. 26, 2012

(54) PHARMACEUTICAL FORMULATIONS

(75) Inventors: DAVID HARRIS, New Providence, NJ (US); Farah J. Munayyer, West Caldwell, NJ (US)

(73) Assignee: Schering Corporation, Kenilworth, NJ (US)

(21) Appl. No.: 13/251,899

(22) Filed: Oct. 3, 2011

Related U.S. Application Data
(63) Continuation of application No. 11/314,597, filed on Dec. 21, 2005, now abandoned.
(60) Provisional application No. 60/638,266, filed on Dec. 22, 2004.

(51) Int. Cl. A61K 31/4545; A61P 37/08

(52) U.S. Cl. 514/290

(57) ABSTRACT

New and improved antihistaminic syrups are disclosed.

### CROSS REFERENCE TO RELATED APPLICATION

This application claims benefit of priority to U.S. Provisional Patent Application 60/638,266, filed Dec. 12, 2004, which is hereby incorporated by reference in its entirety.

### BACKGROUND OF THE INVENTION

The present invention pertains to the field of liquid pharmaceutical formulations, and more particularly to syrup formulations containing antihistamines.

Syrup formulations are commonly used for delivery of pharmacological agents, particularly where the agents are to be delivered to pediatric patients. Traditional syrups are concentrated solutions of sugar (generally sucrose) in purified water, such as Syrup, NF prepared with 850 grams sucrose and sufficient water to make 1000 mL according to the procedure given in the official monograph at page 1990 of NF 19 The National Formulary, United States Pharmacopeial Convention, Inc., Rockville, Md. U.S.A., 2000. However, for purposes of the present invention, the term “syrup” will also encompass those liquid formulations having a sweet taste provided wholly or partly by artificial sweeteners for avoidance of dental and medical problems which may be aggravated by higher caloric sweeteners.

As is well appreciated in the art, syrups frequently are flavored, such as with fruit or mint flavors, usually for purposes of masking an unpleasant taste caused by the presence of a dissolved or suspended pharmacologically active substance. A pleasant taste is particularly important when the formulation is intended for ingestion by children. Typical flavoring agents which are commonly used in sweetened pharmaceuticals, foods, candies, beverages and the like are also useful in the present invention; these materials impart flavors such as grape, cherry, citrus, peach, strawberry, bubble gum, peppermint and many others.

An example of a currently marketed syrup contains 1 mg/mL of the antihistaminic drug loratadine, together with citric acid, artificial flavor, glycerin, propylene glycol, sodium benzoate, sucrose and water; this formulation typically has a pH value between about 2 and 4. However, under certain storage conditions involving contact with the air, losses of loratadine content, and a concomitant generation of impurities, have occurred. Similar problems can occur with formulations containing other, chemically related, drugs, such as desloratadine.

U.S. Pat. No. 6,514,520 discloses an antihistaminic syrup formulation comprising desloratadine and about 0.05 to about 5 mg/mL of an aminopolycarboxylic acid or a salt thereof. However, there still exists a need for new syrup formulations for the delivery of desloratadine and other antihistamines. Accordingly, it is desired to provide a novel storage-stable syrup formulation of desloratadine or related antihistaminic components, which contains only components recognized as being safe for human ingestion, that are sugar free, clear in color and that are storage stable.

### SUMMARY OF THE INVENTION

Accordingly, there is disclosed an antihistaminic syrup formulation comprising desloratadine or a chemically related antihistamine, including any pharmaceutically acceptable salt thereof that is storage stable, at least one pharmaceutically acceptable artificial sweetening agent, at least one pharmaceutically acceptable carrier, wherein the syrup formulation has a pH of greater than about 4.5.

There is also disclosed an antihistaminic syrup formulation comprising desloratadine or a chemically related antihistamine, including any pharmaceutically acceptable salt thereof that is storage stable, at least one pharmaceutically acceptable artificial sweetening agent, at least one pharmaceutically acceptable carrier, wherein the syrup formulation has a pH of about 4.5 to about 6.5.

### DETAILED DESCRIPTION OF THE INVENTION

Where the term “percent” is used herein, it is intended to represent percent by weight, unless the context clearly evidences otherwise.

The compound desloratadine is an antihistaminic active metabolite of loratadine. Desloratadine is a white to off-white powder that is slightly soluble in water, but very soluble in ethanol and propylene glycol. It has an empirical formula: C₁₉H₁₉CIN₂ and a molecular weight of 310.8. The chemical name is 8-chloro-6,11-dihydro-11-(4-piperdinylidene)-5H-benzo[5,6]cyclohepta[1,2-b]pyridine. It is available under the Trade names of Clarinex® and Aerius® from Schering Corp., Kenilworth, N.J.

The antihistaminic syrup formulations of the present invention may also contain one or more other drugs for obtaining more than one therapeutic result from a single dose. Typical drug substances included with desloratadine are sympathomimetic amine decongestants, such as pseudoephedrine, phenylpropanolamine or phenylephrine for relief of the upper airway congestion often accompanying disorders such as rhinitis and upper respiratory infections. Antitussives, such as codeine, hydrocodone or dextromethorphan, for relief from coughing, and expectorants such as guaifenesin, for increasing cough productivity, also are included in combination products. H₃ receptor antagonists may also be used in combination with the syrups of the present invention. The histamine H₃ receptor antagonist may be one or more members selected from the group consisting of thioperamide, impromidine, burimamide, clobenpropit, impentamine, mifetidine, clozapine, S-sopromidine, R-sopromidine, ciproxifam, SKF-91486 (3-(imidazole-4-yl )-propylguanidine sulfate), GR-175737 (Clitherow, et al., (1996) Bioorg. Med. 6: 833-838), GT-2016 (Tedford, et al., (1995) J. Pharm. Exp. Ther 275(2): 596-604), GT-2331 (Tedford, et al., (1998) Eur. J. Pharmacol. 351(3): 307-11), GT-2394 (Yates, et al., (2000) Soc. Neurosci. Abstr. 26: 279.), JB98064 (Linney, et al., (2000) J. Med. Chem. 43: 2362-2370), UCL-1199 (Ganellin, et al., (1995) J. Med. Chem. 38(17): 3342-50), and ABT331440 (PCT Publication No. WO 02/06223).

Other typical agents which may also be included along with desloratadine include non-steroidal anti-inflammatory drugs (NSAIDs), steroids and antiboitics (e.g., antibacterial and antifungal). NSAIDs include aspirin, acetaminophen, phenylpropionic derivatives (e.g., ibuprofen, naproxen), oxicams (e.g., piroxicam), ketorolac, celecoxib and rofecoxib. Steroids included for use in the present invention include mometasone, dexamethasone, butoxicart, rofleponide, budesonide, deflazacort, ciclesonide, fluticasone, beclomethasone, betamethasone, Fluocinolone, prednisone, prednisolone, loteprednol or triamcinolone. Antibacterial agents include β-lactam antibiotics (e.g., pennicillin, amoxicillin, cloxacillin, dicloxacillin, methicillin, nafcillin, oxacillin and piperacillin), aminoglycosides (e.g., amikacin, gentamicin, kanamycin, neomycin, netilmicin, streptomycin and tobramycin), macrolides, lincomycin, and clindamycin, tetracyclines (e.g., demeclocycline, doxycycline, minocycline, oxytetracycline, tetracycline), quinolones (e.g., cinoxacin, nalidixic acid), fluoroquinolones (e.g., iprofloxacin, enoxacin, grepafloxacin, levofloxacin, lomefloxacin, norfloxacin, ofloxacin, sparfloxacin, trovafloxacin), polypeptides (e.g., bacitracin, colistin, polymyxin B), solfonamides, trimethoprim-sulfamethoxazole (TMP-SMX), chloramphenicol, vancomycin, quinupristin/dalfopristin, metronidazole, rifampin, spectinomycin and nitrofurantoin. Antifungals for use in the present invention include posaconazole, voriconazole, ketoconazole, fluconazole, itraconazole, saperconazole, neticonazole, oxiconazole, isoconazole, sulconazole, terconazole, ravuconazole, capsofungin, tioconazole, and/or the pharmaceutically acceptable salts thereof.

Any of these additional ingredients, including salts thereof and other drugs from the same therapeutic classes, are suitable for inclusion in the syrups of the present invention.

Suitable non-sugar based artificial sweetening agents for use in the present invention include sucralose, a flourinated sucrose derivative, saccharin, nutritive dextrose, acesulfame potassium, saccharin and aspartame. Particularly preferred are sucralose and saccharin. The sweetening agent may be present in amounts such as, for instance, about 0.01% to about 10%, preferably about 0.1% to about 1%.

Typically, suitable pharmaceutically acceptable solvents and/or carrier systems include water, alcohols and glycols, especially propylene glycol, sorbitol, ethanol, polyethylene glycol and/or glycerin. The liquid pharmaceutical compositions indicated for pediatric use should be substantially free of and most preferably should not contain ethanol. Use of a combination of at least one of water, propylene glycol, sorbitol and glycerin is preferred. Propylene glycol may be present in a concentration of about 50 to 200 mg/mL. Sorbitol may be present in a concentration of about 100 to 250 mg/mL. Normally, the pharmaceutically acceptable liquid carrier is purified water.

Suitable buffer systems of use in the present invention include, by way of example only, tartaric, fumaric, maleic, phosphoric, and acetic acids and salts. Preferred buffering systems include citric acid and phosphoric acid buffer systems. The citric acid buffer system preferably contains sodium citrate in combination with citric acid. Preferably there is about 0.1 to about 10 grams/liter of sodium citrate, and about 0.05 to about 5 grams/liter of citric acid. Typically suitable buffer systems include those capable of maintaining a pH in the range of greater than about 4.5, preferably about 4.5 to about 6.5, more preferably 5.5.

Suitable thickening agents for use in the present invention include, inter alia, guar gum, gelatin, locust bean gum, tara gum, xanthan gum, tamarind gum, tragacanth gum, karaya gum, konjac mannan, water-soluble carboxyvinyl polymer, sodium carboxymethylcellulose, sodium alginate, pectin, azotobacter vinelandii gum, carrageenan, polyethylene glycol, modified starch, cassia gum, psyllium seed gum, carboxymethylcellulose, hydroxypropyl cellulose, hydroxypropyl methylcellulose, hydroxyethyl cellulose, methyl cellulose and microcrystalline cellulose.

In sugar based syrup formulations it is often desirable to employ antimicrobial preservatives. The amount of a pharmaceutically acceptable preservative required to protect a syrup against microbial growth varies with the proportion of water available for growth, the nature and inherent preservative activity of some formulative materials (as many flavoring oils and co-solvents such as propylene gycol are inherently sterile and possess antimicrobial activity), and the capability of the preservative itself. Among the preservatives commonly used in the preservation of syrups with the usually effective concentrations are benzoic acid (0.1 to 0.2%), sodium benzoate (0.1 to 0.2%), and various combinations of methyl-, propyl-, and butylparabens (totaling about 0.1%). In another aspect of the present invention, it has been found that sodium benzoate is not necessary for certain embodiments of the present invention.

Stabilizers may also be incorporated into the syrup formulation. Useful aminopolycarboxylic acids and salts thereof are those which are safe for ingestion and have sufficient solubility in the syrup formulations to make a stable single phase composition. Commercially available compounds which could be used include iminodiacetic acid, methyliminodiacetic acid, nitrilotriacetic acid, ethylenediaminetetraacetic acid (“EDTA”), diethylenetriaminepentaacetic acid, 1,2-diaminocyclohexane-tetraacetic acid, N-hydroxyethylenediaminetriacetic acid and related compounds. Mixtures of two or more of the foregoing are suitable for use. From the aspects of ready availability, safety, efficacy and cost, the alkali metal salts of EDTA are presently preferred. In those embodiments containing a stabilizer, the stabilizer may be present in amounts of about 0.01 to about 5%, preferably about 0.25%. In an alternative embodiment of the present invention, EDTA is not a necessary ingredient.

Preferably, the formulations of the present invention have less than 0.2% desloratadine degradation products over time under accelerated stability testing, more preferably less than 0.1%. Preferably, the formulations of the present invention are stable at 6 months under accelerated stability testing conditions, more preferably greater than a year, more preferably greater than 15 months and most preferably greater than two years. In addition to being stable, the syrups should not discolor as is known to one of skill in the art.

Most syrups are flavored with synthetic flavorants or with naturally occurring materials such as volatile oils (e.g. orange oil), vanillin, and others, to render the syrup pleasant tasting. Because syrups are aqueous preparations, these flavorants must possess sufficient water-solubility. Typical flavoring agents which are commonly used in sweetened pharmaceuticals, foods, candies, beverages are also useful in the present invention; these materials may impart flavors such as grape, cherry, citrus, peach, strawberry, bubble gum, peppermint and many others are within the scope of the present invention. A preferred flavoring agent is Bubblegum Natural and Artificial #15864, available from Virginia Dare.

Also provided by the invention are methods for treating and/or preventing allergic and inflammatory conditions in humans in need of such treating and/or preventing which comprise administering an effective amount of a desloratadine. The phrase “allergic and inflammatory conditions of the skin or airway passages” as used herein means those allergic and inflammatory conditions and symptoms found on the skin and in the upper and lower airway passages from the nose to the lungs. Typical allergic and inflammatory conditions of the skin or upper and lower airway passages include seasonal and perennial allergic rhinitis, allergic rhinitis associated with cough, non-allergic rhinitis, asthma including allergic and non-allergic asthma, sinusitis, colds, bronchopulmonary conditions of allergic origin associated with cough, where viscosity and mucous adherence are increased, obstructing permeability of the airways, acute, chronic, spasmodic and asthmatic bronchitis, bronchial asthma, bronchiectasis, sinusitis, otitis media, pneumonia; bronchopneumonia, atelectasis by mucous obstruction, and dermatitis, especially allergic and atopic dermatitis, and urticaria and symptomatic dermographism as well as retinopathy, and small vessel diseases, associated with diabetes mellitus.

Prior art syrup formulations of desloratadine oral solution such as that disclosed in U.S. Pat. No. 6,514,520 have been manufactured as follows: Desloratadine and flavor (Natural & artificial flavor for bubblegum, # 15864) are dissolved in propylene glycol. The remaining formulation excipients are dissolved in water. The propylene glycol concentrate is added to the aqueous vehicle with mixing. Water is added qs ad final volume. When the resulting drug product is stored under dark conditions, a strong pink color has been observed to develop over time. This color formation may derive from interaction between desloratadine and the flavorant or between the desloratadine and propylene glycol. It was necessary to add a yellow dye to mask the color change in the prior art syrup formulation. There exists an additional need for novel processes for producing clear and colorless syrups that are sugar free and dye free. As illustrated in the following Examples, the present invention provides syrups that are sugar free and or dye free and that do not substantially discolor over time. Accordingly, the invention will be further described by means of the following examples, which are not intended to limit the scope of the invention as defined by the appended claims.

### EXAMPLE 1

| | Concentration |
|---|---|
| Ingredient | (mg/mL) |
| Desloratadine, | 0.5 |
| micronized | |
| Propylene glycol, | 100 |
| Sorbitol liquid | 150 |
| Citric acid | 0.5 |
| Sodium citrate | 2.83 |
| Hydroxypropyl | 3.5 |
| methylcellulose (E4M) | |
| Edetate disodium | 0.25 |
| Saccharin. | 1.0 |
| Natural & artificial | 0.75 |
| flavor for bubblegum, # | |
| 15864 | |
| Water, purified qs | 1.0 |
| ad | |

To prepare the above syrup formulations, the ingredients with the exception of desloratadine are dissolved or mixed into a vessel as is known to one of skill in the art. The addition to the manufacturing process of the dissolving of the desloratadine directly into the finished formulation that incorporates all of the remaining ingredients listed in the above formula avoids the contact between desloratadine and the propylene glycol and bubble gum flavor solution that may have produced a pink color in the prior art formulations that needed to be color masked with a yellow dye. An exemplary formulation underwent accelerated stability testing to yield the following positive results set forth in Table 1.

TABLE 1

| | | | Disodium | | |
|---|---|---|---|---|---|
| | | | edetate | | |
| Time | | Desloratadine | assay | | |
| point | Storage | assay | (% | Physical | |
| (months) | condition | (% of label) | of label) | appearance | pH |
| Initial | N/A | 104.2 | 100.0 | Clear | 5.5 |
| | | | | colorless | |
| | | | | solution | |
| 3 | Room | 102.8 | 103.2 | Clear | 5.4 |
| | temperature | | | colorless | |
| | | | | solution | |
| 3 | 40° C./75% | 103.2 | 102.8 | Clear | 5.5 |
| | RH | | | colorless | |
| | | | | solution | |
| 6 | Refrigeration | 103.8 | 101.6 | Clear | 5.4 |
| | | | | colorless | |
| | | | | solution | |
| 6 | Room | 103.6 | 100.8 | Clear | 5.4 |
| | temperature | | | colorless | |
| | | | | solution | |
| 6 | 40° C./75% | 103.8 | 100.8 | Clear | 5.5 |
| | RH | | | colorless | |
| | | | | solution | |

As is evident from the data set forth in Table 1, the formulations of the present invention exhibit satisfactory stability performance with respect to desloratadine content and physical appearance under accelerated stability conditions.

### EXAMPLE 2

| | Concentration |
|---|---|
| Ingredient | (mg/mL) |
| Desloratadine, micronized | 0.5 |
| Propylene glycol. | 150 |
| Sorbitol liquid | 150 |
| Citric acid | 0.5 |
| Sodium citrate | 1.26 |
| Edetate disodium | 0.25 |
| Hydroxypropyl methylcellulose | 3.5 |
| (E4M) | |
| Sucralose | 4.0 |
| Natural & artificial flavor for | 0.75 |
| bubblegum, # 15864 | |
| Water, purified, qs ad | 1.0 |

The ingredients were prepared in accordance with the procedure set forth in Example 1 above. The exemplary formulation underwent accelerated stability testing to yield the following results set forth in Table 2.

TABLE 2

| | | Desloratadine | |
|---|---|---|---|
| Time | | assay | |
| (months) | Storage condition | (% of label) | Appearance |
| Initial | — | 99.6 | Clear colorless solution |
| 1 | 25° C./60% RH | 98.6 | Clear colorless solution |
| | 40° C./75% RH | 99.0 | Clear colorless solution |
| | 55° C. | 97.6 | Clear colorless solution |
| 3 | 25° C./60% RH | 97.7 | Clear colorless solution |
| | 40° C./75% RH | 97.1 | Clear colorless solution |
| 5½ | 25° C./60% RH | 97.7 | Clear colorless solution |
| | 40° C./75% RH | 97.4 | Clear colorless solution |

As is evident from the data set forth in Table 2, the exemplary formulation of the present invention exhibits satisfactory stability performance with respect to desloratadine content and physical appearance under accelerated stability conditions.

These syrups have exhibited a storage stability of 15 months.

### EXAMPLE 3

| | Temp | | | Physical |
|---|---|---|---|---|
| Age | ° C. | DL Assay % | Edetate % | Observation |
| 1 month | 25 C./60% RH | 98.6 | 97.4 | Clear |
| | 55° C. | 97.6 | 94.8 | Clear |
| | 25 C./60% RH | 97.6 | Absent | Clear |
| | 50° C. | 97.7 | Absent | Clear |

Two batches of syrups were prepared. One batch of syrup contained EDTA and the other batch of syrup did not. Both syrups produced a clear colorless solution when subjected the storage conditions set forth in the above table.

Having described specific preferred embodiments of the invention with reference to the accompanying drawings, it will be appreciated that the present invention is not limited to those precise embodiments and that various changes and modifications can be effected therein by one of ordinary skill in the art without departing from the scope or spirit of the invention as defined by the appended claims.

What is claimed is:

1. An antihistaminic syrup formulation comprising desloratadine or a pharmaceutically acceptable salt thereof that is storage stable, at least one pharmaceutically acceptable artificial sweetening agent, at least one pharmaceutically acceptable carrier, wherein the syrup formulation has a pH of greater than about 4.5.

2. The antihistaminic syrup formulation according to claim 1 further comprising a buffering system, wherein the buffering system comprises sodium citrate and citric acid.

3. The antihistaminic syrup formulation according to claim 2, wherein the buffering system comprises sodium citrate and citric acid and wherein the sodium citrate is present in a concentration of about at least 0.1 mg/mL and the citric acid is present in a concentration of about at least 0.1 mg/mL.

4. The antihistaminic syrup formulation according to claim 1, wherein the desloratadine is present in a concentration of about 0.1 to about 10 mg/L.

5. The antihistaminic syrup formulation according to claim 4, wherein the desloratadine is present in a concentration of about 0.5 mg/mL.

6. The antihistaminic syrup formulation according to claim 1, wherein there is at least one pharmaceutically acceptable sweetening agent selected from the group consisting of sucralose, saccharin, a fluourinated sucrose derivative, acesulfame potassium and aspartame.

7. The antihistaminic syrup formulation according to claim 1, wherein the at least one pharmaceutically acceptable carrier is selected from the group consisting of water, propylene glycol, polyethylene glycol, sorbitol and glycerin and combinations of two or more thereof.

8. The antihistaminic syrup formulation according to claim 1 further comprising at least one pharmaceutically acceptable viscosity increasing agent.

9. The antihistaminic syrup formulation according to claim 1, wherein the at least one pharmaceutically acceptable viscosity increasing agent is selected format least one of the group consisting of guar gum, gelatin, locust bean gum, tara gum, xanthan gum, tamarind gum, tragacanth gum, karaya gum, konjac mannan, water-soluble carboxyvinyl polymer, sodium carboxymethylcellulose, sodium alginate, pectin, azotobacter vinelandii gum, carrageenan, polyethylene glycol, modified starch, cassia gum, psyllium seed gum, carboxymethylcellulose, hydroxypropyl cellulose, hydroxypropyl methylcellulose, hydroxyethyl cellulose, methyl cellulose and microcrystalline cellulose.

10. The antihistaminic syrup formulation according to claim 1, wherein the liquid formulation is storage stable for at least 15 months.

11. The antihistaminic syrup formulation according to claim 1 further comprising about 0.05 to about 5 mg/mL of an aminopolycarboxylic acid or a salt thereof.

12. An antihistaminic syrup formulation comprising desloratadine or a chemically related antihistamine, including any pharmaceutically acceptable salt thereof that is storage stable, at least one pharmaceutically acceptable artificial sweetening agent, at least one pharmaceutically acceptable carrier, wherein the syrup formulation has a pH of about 4.5 to about 6.5.

13. The antihistaminic syrup formulation according to claim 12 further comprising a buffering system.

14. The antihistaminic syrup formulation according to claim 13, wherein the buffering system comprises sodium citrate and citric acid and wherein the sodium citrate is present in a concentration of about at least 0.1 mg/mL and the citric acid is present in a concentration of about at least 0.1 mg/mL.

15. The antihistaminic syrup formulation according to claim 12, wherein the desloratadine is present in a concentration of about 0.1 to about 10 mg/mL.

16. The antihistaminic syrup formulation according to claim 15, wherein the desloratadine is present in a concentration of about 0.5 mg/mL.

17. The antihistaminic syrup formulation according to claim 13, wherein there is at least one pharmaceutically acceptable sweetening agent selected from the group consisting of sucralose, a fluourinated sucrose derivative, dextrose, acesulfame potassium, saccharin and aspartame.

18. The antihistaminic syrup formulation according to claim 12, wherein the at least one pharmaceutically acceptable carrier is selected from the group consisting of water, propylene glycol, polyethylene glycol, sorbitol and glycerin and any combinations of two or more thereof.

19. The antihistaminic syrup formulation according to claim 12 further comprising at least one pharmaceutically acceptable viscosity increasing agent.

20. The antihistaminic syrup formulation according to claim 19, wherein the at least one pharmaceutically acceptable viscosity increasing agent is selected from at least one of the group consisting of guar gum, gelatin, locust bean gum, tara gum, xanthan gum, tamarind gum, tragacanth gum, karaya gum, konjac mannan, water-soluble carboxyvinyl polymer, sodium carboxymethylcellulose, sodium alginate, pectin, azotobacter vinelandii gum, carrageenan, polyethylene glycol, modified starch, cassia gum, psyllium seed gum, carboxymethylcellulose, methyl cellulose, hydroxypropyl cellulose, hydroxypropyl methylcellulose, hydroxyethyl cellulose, methyl cellulose and microcrystalline cellulose.

21. The antihistaminic syrup formulation according to claim 12, wherein the liquid formulation is storage stable for at least 15 months.

22. The antihistaminic syrup formulation according to claim 12 further comprising about 0.05 to about 5 mg/mL of an aminopolycarboxylic acid or a salt thereof.
